(19) United States
(12) Patent Application Publication
(10) Pub. No.: US 2008/0011293 A1
(43) Pub. Date: Jan. 17, 2008

(54) Stable Xylometazoline and Oxymetazoline Solution

(76) Inventor: Frieder Maerz, Soergenloch (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/840,778

(22) Filed: Aug. 17, 2007

Related U.S. Application Data
(63) Continuation of application No. 10/768,768, filed on Jan. 30, 2004.
Application No. 10/768,768 is a continuation of application No. 09/337,789, filed on Jun. 22, 1999, now abandoned.

(51) Int. Cl. A61M 11/00; A61K 33/38; A61P 11/00

(52) U.S. Cl. 128/200.22; 424/618

(57) ABSTRACT

The present invention relates to a biologically and chemically stable xylometazoline and/or oxymetazoline solution containing glycerol and/or sorbitol as adjuvant.

This application claims the benefit under 35 U.S.C. 120 of U.S. patent application Ser. No. 10/768,768, filed Jan. 30, 2004, and U.S. patent application Ser. No. 09/337,789, filed Jun. 22, 1999, both of which prior-filed application are incorporated herein by reference in their entirties.

The present invention relates to a biologically and chemically stable xylometazoline and/or oxymetazoline solution containing glycerol and/or sorbitol as adjuvant.

### BACKGROUND OF THE INVENTION

Xylometazoline [2-(4-tert.butyl-2,6-dimethylbenzyl)-4,5-dihydro-1-H— -imidazole], like oxymetazoline [6-tert.butyl-3-(4,5-dihydro-1-H-imidazol-2-ylmethyl)-2,4-dimethphenyl], is a vasoconstrictor from the class of imidazole active substances. Both can be used as rhinological agents. When used as rhinological agents the substances are administered in the form of an aqueous solution using a nasal spray pump. As it is known that the free bases xylometasoline and oxymetazoline have only limited stability to hydrolysis in aqueous solution when used for pharmaceutical purposes, the active substance is generally only used in the form of a salt, particularly the hydrochloride.

Sprayable rhinological agents containing xylometazoline or oxymetazoline in which the active substance does not occur as the hydrochloride are disclosed in WO 88/00473. According to this specification, xylometazoline may be used as a free base to form an anhydrous formulation together with an ethereal oil in a triglyceride with no other stabiliser.

Preservatives are added to rhinological solutions containing xylometasoline and oxymetazoline hydrochloride. These preservatives prevent contamination with bacteria and other microorganisms during the storage and use of the solution. Preservatives are needed particularly when these formulations contain other ingredients which promote the growth of microorganisms. Such ingredients might be, for example, buffers based on citric acid, lactic acid, propionic acid, etc. or adjuvants or other compounds. The adjuvants used in formulations containing xylometazoline or oxymetazoline are usually polyvinylpyrrolidone, polysorbate, various cellulose derivatives and/or polyalcohols such as glycerol and sorbitol. Aqueous solutions with small amounts of sorbitol and/or glycerol are particularly known to form a very good nutrient medium for microorganisms (M. Barr, L. F. Tice, Journal of the American Pharmaceutical Association, Scientific Edition, 46(4), 1957, 217-218]. Therefore, preservatives have to be added particularly to pharmaceutical solutions which contain glycerol or sorbitol [M. Barr, L. F. Tice, Journal of the American Pharmaceutical Association, Scientific Edition, 46(4), 1957, 221-222]. The preservatives investigated by the authors include sodium benzoate, benzoic acid, methylparaben, ethylparaben, propylparaben, butylparaben, cetyl pyridinium chloride, benzethonium chloride, sodium dehydroacetate, saligenin, sorbic acid, benzalkonium chloride, etc.

It is worth mentioning in this context that there has been discussion in the literature, with regard to aqueous solutions containing a large amount of glycerol and sorbitol, of converting the effect which promotes the growth of microorganisms into an opposite effect [H. P. Fiedler, Lexikon der Hilfsstoffe fur Pharmazie, Kosmetik und angrenzende Gebiete, Editio Cantor Verlag, 4^{th} Edition, p. 1424]. According to M. Barr and L. F. Tice this inhibitory effect of more highly concentrated glycerol or sorbitol solutions occurs, inter alia, as a function of the pH of the solution and the biological species in question. Thus, the authors observe that the inhibitory effect of glycerol for the most sensitive species Pseudomonas aeruginosa only sets in above 30% by weight at a pH of 7.4 [M. Barr, L. F. Tice, Journal of the American Pharmaceutical Association, Scientific Edition, 46(4), 1957, 217-218], or above 25% by weight at a pH of 5.6 adjusted using HCl and NaOH [M. Barr, L. F. Tice, Journal of the American Pharmaceutical Association, Scientific Edition, 46(4), 1957, 219-221]. For sorbitol the values are identical in neutral but under acid conditions 40% by weight are required to trigger the inhibitory effect on P. aeruginosa [M. Barr, L. F. Tice, Journal of the American Pharmaceutical Association, Scientific Edition, 46(4), 1957, 221-222]. These observations relating to glycerol are backed up by other authors in a similar manner [E. Mariani, C. J. Libbey, W. Litsky, Development in industrial Microbiology 14 1973, 356-3601]. In rhinological solutions the quantity of sorbitol or glycerol is always below this antimicrobially active amount, i.e., within the range which may promote the growth of microorganisms.

However, preservatives have various disadvantages, especially in rhinological agents. They may not only damage the defence mechanisms of the nasal mucosa, phagocytosis, chemotaxis and the mucociliary transport system, but may also cause cell damage, allergic reactions and other irritations. On the other hand, formulations from which preservatives are omitted can be expected to suffer considerable microbiological contamination during storage or use, particularly if the formulation contains other ingredients which promote the growth of microorganisms.

### DESCRIPTION OF THE INVENTION

The aim of the present invention is to provide an isotonic formulation of a solution containing an imidazole active substance which overcomes the drawbacks known from the prior art.

A further objective of the invention is to formulate an isotonic solution containing an imidazole active substance as a rhinological agent, which contains only a minimum amount of other additives so as to reduce irritation of the nasal mucosa as far as possible.

The invention also sets out to formulate a rhinological agent containing an imidazole active substance and a polyalcohol as adjuvant, the resulting solution containing no or virtually no other substances which promote the growth of microorganisms.

It should be noted at this point that in the context of this invention there is no distinction made between “bacteriostatic” and “bactericidal” etc. Instead, these effects are subsumed under concepts such as “not a suitable nutrient medium for microorganisms”, “a negative influence on the growth of microorganisms” or “antibacterial action/effect”, “no susceptibility to microbial contamination”, etc., without any further differentiation.

Surprisingly, it has now been found that neutral to slightly acidic isotonic solutions buffered with inorganic buffers comprising one or both of the two imidazole active substances xylometazoline and/or oxymetazoline hydrochloride, which contains sorbital and/or glycerol in an amount of less than 10% by weight, do not form a suitable nutrient medium for microorganisms but rather may even negatively influence the growth of microorganisms.

The present invention achieves the objectives set by providing a stable formulation of a solution containing xylometazoline and/or oxymetazoline as active substance, containing the active substance, a solvent such as water which is pharmaceutically acceptable for nasal administration, an adjuvant selected from among sorbitol and/or glycerol and an inorganic pH buffer.

The formulation is made isotonic.

One advantage of the formulation according to the invention is that there is no need to use conventional preservatives such as, for example, benzalkonium chloride, chlorhexidine gluconate, benzyl alcohol, disodium ethylenediamine tetraacetate or thimerisol.

It is critical, however, that the formulation is such that it does not promote contamination with microorganisms which leads to the accumulation of microorganisms in the formulation during the storage or usage period beyond a level which is pharmaceutically acceptable.

Owing to the fact that the above-mentioned preservatives are not needed in the formulation, the problems known from the prior art which arise from the use of preservatives in rhinological formulations are overcome.

Isotonic solutions containing xylometazoline and/or oxymetazoline as active substance, sorbitol and/or glycerol, the substances required to achieve an isotonic solution and an inorganic buffer which contain no other additives but are yet immune from contamination by microorganisms to a level which is pharmaceutically unacceptable, are not known.

The concentration of the xylometazoline and/or oxymetazoline, or the hydrochlorides thereof, is within the range appropriate thereto for nasal administration for each of these active substances, preferably in a concentration of between 0.01 and 1.0% by weight, more preferably between 0.01 and 0.5% by weight and most preferably between 0.05 and 0.1% by weight.

The solvents may be any pharmaceutically acceptable solvents for nasal use such as water or an ethanol/water mixture. The preferred solvent is water.

The adjuvant used may be sorbitol, glycerol or a mixture of both. Preferably, either sorbitol or glycerol is used. The job of this adjuvant is, on the one hand, to improve the solubility of the active substance in the solvent and, on the other hand, to act as a moistening agent to prevent the nasal mucosa from drying out.

In one embodiment of the invention the proportion of the adjuvant is from 1 to 10% by weight, preferably 2 to 6% by weight. For sorbitol the proportion is most preferably 3.5 to 4.5% by weight, especially 4.0% by weight, for glycerol the amount is preferably 2.0 to 2.8% by weight, most preferably 2.4% by weight.

In another formulation of this kind a buffer system is used to provide a pH of from 4.0 to 7.5. Preferably, the pH is set at 5.0 to 6.8, more preferably to 5.5 to 6.8, most preferably to 5.8 to 6.0. Pharmaceutically acceptable inorganic buffers are used for this purpose. Buffers based on inorganic alkali metal phosphates and alkali metal borates are preferred, particularly the corresponding sodium and/or potassium salts. Buffers based on monosodium dihydrogen-disodium monohydrogen phosphate and/or the analogous potassium salts are most particularly preferred. If desired, the pH may be corrected by further adding hydrochloric acid and/or sodium hydroxide solution.

Surprisingly, it has also been found that the negative effect on the growth of microorganisms is intensified if the formulation is administered by means of a spray or inhaler which has components made of oligodynamically active metals such as silver between the active substance reservoir and the sprayhead. A spray device of this kind is disclosed, for example, in WO 97/18902, to which reference is expressly made hereby. By oligodynamic substances is meant metals or metal ions with a germicidal effect. These include silver or copper, for example.

Interestingly, in these cases, the more intensive antimicrobial effect occurs both when the oligodynamically active substance can be detected in the formulation after some time and also when the oligodynamic substance cannot be detected in a spray of this kind after storage and use.

Therefore, the invention also relates to solutions of the kind described above containing xylometazoline and/or oxymetazoline, which additionally contain an oligodynamically effective substance such as silver in pharmaceutically acceptable amounts. Formulations of this kind are unknown. Apart from the active substance, the adjuvant described above and inorganic buffer, the formulation does not contain any other organic additives, particularly none such as citric acid, other organic acids or their salts, for example.

The formulation as described is suitable for use as a rhinological agent.

### EXAMPLES

The invention will be illustrated in more detail hereinafter by means of some investigations into biological stability.

Investigations of biological stability are carried out on the basis of tests for adequate preservation (EuAB 1997, 5.1.3). 990 μl of each of the formulations to be investigated are inoculated according to the provisions of EuAB 1997 with 10 μl of a pathogen solution corresponding to a quantity of about 10⁵ to 10⁶ colony-forming units (CFU) mlg. The resulting solution is stored for 14 days at room temperature and throughout the entire period the change in the number of pathogens is determined at specific times.

The inoculating pathogen solution is obtained from cultures which are 18 to 24 hours old (in the case of bacteria) or a few days old (in the case of fungi) in physiological saline solution.

The test organisms used are E. coli ATCC 8739, Ps. aeruginosa ATCC 9027 and St. aureus ATCC 6538P.

The number of pathogens is determined by taking 50 .μl samples at times t=0 h, 6 h, 24 h, 7 days and 14 days. From these a dilution series is produced in physiological saline. The dilutions are transferred to agar dishes so that after a suitable incubation period the number of vital pathogens can be determined.

For each of the formulations being investigated, a second parallel investigation is carried out differing from the one described above in that a silver thread is immersed in the test solution (1 ml) inoculated with the pathogens.

### Example 1

Investigation of the following 0.05% by weight xylometazoline solution with a pH of 6.0 for biological stability:

mg/10 ml=10150 mg

| | |
|---|---|
| (01) Xylometazoline hydrochloride | 5.0 |
| (02) Sorbitol | 400.0 |
| (03) Monosodium dihydrogen phosphate dehydrate | 40.0 |
| (04) Disodium monohydrogen phosphate dehydrate | 6.5 |
| (05) Water, purified | 9698.5 |

If desired, the pH is corrected by the addition of 1 N hydrochloric acid and/or 1 N sodium hydroxide solution.

The results show that the formulation does not constitute a suitable nutrient medium for growth for any of the test organisms described, but rather the number of microorganisms is reduced significantly compared with the inoculum. The results are shown in Table 1, which indicates the growth of microorganisms in isotonic formulations with 0.05% by weight of xylometasoline. Under the heading xylometazoline are the results for the solution investigated without a silver thread, whereas under the heading xylometazoline+silver are given the results for the solutions containing silver threads.

TABLE 1
Growth of microorganisms in isotonic formulations
containing 0.05% by weight of xylometazoline*

| | | |
|---|---|---|
| | | Xylometazoline + |
| Time | Xylometazoline | silver |

Tab. 1a: Test Organism: E. coli ATCC 8739

| | | |
|---|---|---|
| 0 h | 0 | 0 |
| 6 h | −0.95 | −0.44 |
| 24 h | −2.70 | −2.30 |
| 7 d | −0.29 | <−4.26 |
| 14 d | −1.96 | <−4.26 |
| N(0) | 5.85 | 5.56 |

Tab. 1b: Test Organism: Ps. aeruginosa ATCC 9027

| | | |
|---|---|---|
| 0 h | 0 | 0 |
| 6 h | −1.25 | −0.44 |
| 24 h | −2.70 | −2.30 |
| 7 d | −0.29 | <−4.26 |
| 14 d | −1.96 | <−4.26 |
| N(0) | 5.85 | 5.56 |

Tab. 1c: Test Organism: St. aureus ATCC 6539P

| | | |
|---|---|---|
| 0 h | 0 | 0 |
| 6 h | −0.23 | −0.13 |
| 24 h | −0.30 | −2.64 |
| 7 d | −2.76 | <−4.55 |
| 14 d | −3.91 | <−4.55 |
| N(0) | 6.40 | 5.85 |

*Vital count, expressed as logarithm of the difference in the vital count between the sample and the inoculum N(0).

### Example 2

Investigation of the following 0.05% by weight xylometazoline solution with a pH of 6.0 for biological stability:

mg/10 ml=10150 mg

| | |
|---|---|
| (01) Xylometazoline hydrochloride | 10.0 |
| (02) Sorbitol | 400.0 |
| (03) Monosodium dihydrogen phosphate dehydrate | 39.5 |
| (04) Disodium monohydrogen phosphate dehydrate | 6.6 |
| (05) Water, purified | 9693.9 |

If necessary the pH is corrected by the addition of 1 N hydrochloric acid and/or 1 N sodium hydroxide solution.

The results show that the formulation does not constitute a suitable nutrient medium for growth for any of the test organisms described, but rather the number of microorganisms is reduced significantly compared with the inoculum. The results are shown in Table 2, which indicates the growth of microorganisms in isotonic formulations with 0.1% by weight of xylometasoline. Under the heading xylometazoline are the results for the solution investigated without a silver thread, whereas under the heading xylometazoline+silver are given the results for the solutions containing silver threads.

TABLE 2
Growth of microorganisms in isotonic formulations
containing 0.1% by weight of xylometazoline*

| | | |
|---|---|---|
| | | Xylometazoline + |
| Time | Xylometazoline | silver |

Tab. 1a: Test Organism: E. coli ATCC 8739

| | | |
|---|---|---|
| 0 h | 0 | 0 |
| 6 h | −1.84 | −2.83 |
| 24 h | −3.58 | −4/40 |
| 7 d | <−4.12 | <−4.40 |
| 14 d | <−4.12 | <−4.40 |
| N(0) | 5.48 | 5.70 |

Tab. 1b: Test Organism: Ps. aeruginosa ATCC 9027

| | | |
|---|---|---|
| 0 h | 0 | 0 |
| 6 h | −1.78 | <−4.30 |
| 24 h | −2.70 | <−4.30 |
| 7 d | <−4.34 | <−4.30 |
| 14 d | <−4.34 | <−4.30 |
| N(0) | 5.64 | 5.60 |

Tab. 1c: Test Organism: St. aureus ATCC 6539P

| | | |
|---|---|---|
| 0 h | 0 | 0 |
| 6 h | −0.34 | −3.27 |
| 24 h | −1.03 | −4.73 |
| 7 d | <−4.21 | <−4.73 |
| 14 d | <−4.21 | <−4.73 |
| N(0) | 5.51 | 6.03 |

*Vital count, expressed as logarithm of the difference in the vital count between the sample and the inoculum N(0).

What is claimed is:

1. In an inhaler for nasal administration which comprises a reservoir holding a stable, isotonic formulation having a pH of 4.5 to 7.5 with xylometazoline hydrochloride or oxymetazoline hydrochloride or both as active substance, which formulation further comprises an adjuvant selected from sorbitol or glycerol or both and a buffer selected from an inorganic pH buffer or trometamol, and a sprayhead for nasal administration, the improvement which comprises an oligodynamically active metal or metal ions in the region between the reservoir and the sprayhead through which the formulation passes for administration, or within the reservoir.

2. The inhaler as recited in claim 2, wherein the formulation further comprises hydrochloric acid or sodium hydroxide solution or both.

3. The inhaler as recited in claim 2, wherein the formulation comprises sodium phosphate buffer, potassium phosphate buffer, sodium borate buffer, potassium borate buffer, or a mixture of one or more of such buffers.

4. The inhaler as recited in claim 2, wherein the formulation comprises a monosodium dihydrogen-disodium monohydrogen phosphate buffer, monopotassium dihydrogen-dipotassium monohydrogen phosphate buffer, or a mixture of such buffers.

5 . The inhaler as recited in claim 2, wherein the formulation has a pH of 5.0 to 7.2.

6. The inhaler as recited in claim 2, wherein the formulation has a pH of 5.5 to 6.8.

7. The inhaler as recited in claim 2, wherein the formulation has a pH of 5.8 to 6.0.

8. The inhaler as recited in claim 2, wherein the formulation has a pH of 6.1 to 6.3.

9. The inhaler as recited in claim 2, wherein the active substance is present in a concentration of between 0.01 and 1.0% by weight in the formulation.

10. The inhaler as recited in claim 2, wherein the active substance is present in a concentration of between 0.01 and 0.5% by weight in the formulation.

11. The inhaler as recited in claim 2, wherein the active substance is present in a concentration of between 0.05 and 0.1% by weight in the formulation.

12. The inhaler as recited in claim 2, wherein the formulation further comprises water as solvent.

13. The inhaler as recited in claim 2, wherein the formulation further comprises a mixture of ethanol and water as solvent.

14. The inhaler as recited in claim 2, wherein the proportion of adjuvant in the formulation is 1 to 10% by weight.

15. The inhaler as recited in claim 2, wherein the proportion of adjuvant in the formulation is 2 to 6% by weight.

16. The inhaler as recited in claim 2, wherein the proportion of adjuvant in the formulation is 3.5 to 4.5% by weight, and the adjuvant is sorbitol.

17. The inhaler as recited in claim 2, wherein the proportion of adjuvant in the formulation is 4.0% by weight and the adjuvant is sorbitol.

18. The inhaler as recited in claim 2, wherein the proportion of adjuvant in the formulation is 2.0 to 2.8% by weight, and the adjuvant is glycerol.

19. The inhaler as recited in claim 2, wherein the proportion of adjuvant in the formulation is 2.4% by weight and the adjuvant is glycerol.

20. The inhaler as recited in claim 2, wherein the oligodynamically active metal or oligodynamically active metal ions is silver or are silver ions.
